SEQUENCE LISTING (1 sequence)

SEQ ID NO: 1 - protein, 354 aa, human VEGF-D
MYREWVVVNVFMMLYVQLVQGSSNEHGPVKRSSQSTLERSEQQIRAASSLEELLRTTHSEDWKLWRCRLRLKSFTSMDSRSASHRSTRFAATFYDIETLKVIDEEWQRTQCSPRETCVEVASELGKSTNTFFKPPCVNVERCGGCCNEESFMCMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPDMMFDEDRCECVCKTPCPKDLIQHPKNCSCFECKESEETCCQKHKLFHPDTCSCEDRCPFHTRPCASGKTACAKHCRFPKEKRAAQGPHSRKNP

(19) United States
(12) Patent Application Publication
(10) Pub. No.: US 2003/0166547 A1
(43) Pub. Date: Sep. 4, 2003

(54) VEGF-D and angiogenic use thereof

(75) Inventor: Salvatore Oliviero, Siena (IT)

Correspondence Address: Chiron Corporation, Intellectual Property, P.O. Box 8097, Emeryville, CA, 94662-8097, US

(73) Assignee: Chiron Corporation, Emeryville, PA

(21) Appl. No.: 10/174,930

(22) Filed: Jun. 19, 2002

Related U.S. Application Data
(63) Continuation of application No. 09/640,171, filed on Aug. 15, 2000, now abandoned.
(60) Provisional application No. 60/149,300, filed on Aug. 16, 1999.

(51) Int. Cl. A61K 38/18

(52) U.S. Cl. 514/12

(57) ABSTRACT

The present invention is directed to a method of inducing angiogenesis in a tissue in need of angiogenesis, comprising providing the tissue in need of angiogenesis with an angiogenically effective amount of a recombinant c-fos induced growth factor/vascular endothelial growth factor D (Figf/Vegf-D), which is secreted factor of the VEGF family which binds to the vessel and lymphatic receptors VEGFR-2 and VEGFR-3 (VEGF-D). In another aspect, the present invention is directed to a method of inducing angiogenesis in an area in need of angiogenesis in a mammal comprising administering to said area in need of angiogenesis an angiogenically effective amount VEGF-D. The VEGF-D is provided or administered in solution of slow release form.

### I. BACKGROUND

1. Field of the Invention

The present invention is directed to the use of Figf/VEGF-D or angiogenic fragments or muteins thereof as an angiogenic agent. More specifically, the present invention is directed to a method for inducing angiogenesis in vitro or in vivo comprising administering an effective amount of VEGF-D or an angiogenically active fragment or mutein thereof. The present invention is useful because it provides a method for inducing angiogenesis (or neovascularization) in a patient in need thereof. It also provides a method for treating various ischemic conditions manifest by vascular insufficiency, such as peripheral vascular disease, coronary artery disease or myocardial infarction.

2. Background

Vascular endothelial growth factor (VEGF) is a term that was originally used to refer to a single endothelial cell-specific mitogen that was structurally related to platelet derived growth factor. See Leung et al., “Vascular Endothelial Growth Factor Is a Secreted Mitogen,” Science, 246:1306-1309 (1989); and Tischer, et al., “The Human Gene for Vascular Endothelial Growth Factor,” J. Biol. Chem., 266(18):11947-11954 (1991). However, analysis of the cDNA clones for VEGF predicted the existence of the 189-, 165-, and 121-residue isoforms. Tischer at page 11947. Subsequent to the discovery of the VEGF gene, the predicted isoforms of VEGF were found as were several VEGF homologues.

Today, the term VEGF is not only used to refer to the original VEGF protein, but also to a family of basic, homodimeric proteins that are homologous to VEGF. The members of the VEGF family are designated as VEGF (or VEGF-A), VEGF-B, VEGF-C and VEGF-D. For clarity, the first member of the family, VEGF, will be referred to herein as VEGF-A. The VEGF family of proteins is characterized by having a highly conserved central region, characterized by the invariant presence in homologous positions of 15 cysteine residues, 8 of which are involved in intra- and intermolecular disulfide bonding. See Ferrara, et at., “The Biology of Vascular Endothelial Growth Factor,” Endocrine Reviews, 18(1):4-25 (1997) at FIG. 4. As a result, the four VEGF homologues have a similar shape (tertiary structure) and are capable of spontaneously forming heterodimers when coexpressed. The homologous positioning of 8 of the 15 conserved cysteine residues of VEGF correspond to the 8 conserved cysteine residues of the PDGF family as comparatively shown in e.g., WO 98/02543 at FIG. 3; and Keck, et al., “Vascular Permeability Factor, an Endothelial Cell Mitogen Related to PDGF,” Science 246:1309-1312 (1989) at page 1311, col. 2 and FIG. 4.

Human VEGF-A exists in four isoforms, having 121, 165, 189 and 206 amino acids, respectively. These four isoforms are designated as VEGF-A₁₂₁, VEGF-A₁₆₅, VEGF-A₁₈₉, and VEGF-A₂₀₆, respectively. See Ferrara, et al., “The Biology of Vascular Endothelial Growth Factor,” Endocrine Reviews, 18(1):4-25 (1997) at page 5. The human VEGF-A gene is organized into eight (8) exons separated by seven (7) introns and its coding region spans 14 kb. Id. Alternative exon splicing of the single VEGF-A gene accounts for all of the heterogeneity. VEGF-A₁₆₅ lacks the residues encoded by exon 6, while VEGF-A₁₂₁ lacks the residues encoded by exons 6 and 7. Id. The three shorter isoforms of VEGF-A are based upon VEGF-A₂₀₆ and reflect splice variations that occur in the carboxy half of the molecule. However, the last six amino acids (exon 8) of the carboxy terminus are conserved in all four splice variants.

The cDNA sequences that encode human VEGF-A₁₂₁ and human VEGF-A₁₆₅ and their deduced amino acid sequences are well-known in the art. See Leung, et al., “Vascular endothelial growth factor is a secreted angiogenic mitogen,” Science 246:1306-1309 (1989) at FIG. 2B as described at page 1307, col. 3. Likewise, the cDNA sequence and the deduced amino acid sequence for human VEGF-A₁₈₉ are well-known in the art. See Keck, et al., “Vascular Permeability Factor, an Endothelial Cell Mitogen Related to PDGF,” Science, 246:1309-1312 (1989); see also Tischer et al., “The human gene for vascular endothelial growth factor,” J. Biol. Sci., 266:11947-11954 (1991). Finally, the cDNA sequence and deduced amino acid sequence for VEGF-A₂₀₆ are also well-known in the art. See Houck, et al., “The vascular endothelial growth factor family: identification of a fourth molecular species and characterization of alternative splicing of RNA,” Mol. Endocrinol. 5: 1806-1814 (1991) at FIG. 2A.

An overlapping comparison of the amino acid sequences of the four splice variants (isoforms) of VEGF-A is shown in Ferrara, et al., “Molecular and Biological Properties of the Vascular Endothelial Growth Factor Family of Proteins,” Endocrine Reviews 13(1):18-32 (1992) at page 21, FIG. 1. The shortest isoform, VEGF-A₁₂₁, which is freely soluble in the extracellular milieu, is slightly acidic due to the absence of most of the carboxy terminus (i.e., exons 6 and 7) which are rich in basic amino acid residues. The longer isoforms, VEGF-A₁₆₅, VEGF-A₁₈₉, and VEGF-A₂₀₆, are less soluble, and thus, less diffusible, than VEGF-A₁₂₁, but exhibit both a mitogenic activity and a binding affinity for a heparin-rich matrix that increases with increasing length at the carboxy terminus. By way of example, VEGF-A₁₆₅ is more than 100-fold more mitogenic than the shorter, more soluble and more acidic VEGF-A₁₂₁. See Carmeliet et al., “Vascular development and disorders: Molecular analysis and pathogenic insights,” Kidney International, 53:1519-1549 (1998) at pages 1521-1522. Thus, while all VEGF-A isoforms exhibit mitogenic activity, the amount of activity, the highly basic and heparin binding carboxy terminus of VEGF-A is important to maximizing activity, reducing solubility and reducing diffusibility. Although the mechanism by which VEGF-A stimulates angiogenesis is not known, Banai suggests that VEGF-A promotes angiogenesis in part via stimulation of endothelial release of PDGF. Banai, et al., “Angiogenic-Induced Enhancement of Collateral Blood Flow to Ischemic Myocardium by Vascular Endothelial Growth Factor in Dogs,” Circulation, 89(5):2183-2189 (May 1994). However, not all of the VEGF proteins exhibit the same activity due to their structural differences and their ability to bind to different VEGF receptors. For example, VEGF-A binds to VEGF receptor-1 (VEGFR-1 or FLT1) and to VEGF receptor-2 (VEGFR-2 or FLK1), but not to VEGF receptor-3 (VEGFR-3 or Flt4). See e.g., Ferrara, et al., (1997) at page 12; also Joukov, et al., (1996) at page 296.

Human VEGF-B, which is found in abundance in heart and skeletal muscle, is a known nonglycosylated, highly basic heparin binding protein that has the amino acid sequence shown in FIG. 1 of Olofsson, et al., “Vascular endothelial growth factor B, a novel growth factor for endothelial cells,” PNAS USA 93:2576-2581 (1996). According to Olofsson, VEGF-B is coexpressed with VEGF-A. Id at page 2576 (Abstract). Like the VEGF-As, VEGF-B is expressed as a prohormone and has 188 amino acid residues of which residues 1-21 are a putative leader sequence and thus are not necessary for angiogenic activity. Id. at page 2577, col.2. Hence, mature human VEGF-B comprises the 167 residues that follow the putative leader sequence. Id. The human prohormone VEGF-B also has 88% sequence identity to murine prohormone VEGF-B, varying at residue positions 12, 19, 20, 26, 28, 30, 33, 37, 43, 57, 58, 63, 65, 105, 130, 140, 144, 148, 149, 165, 168, 186 and 188 in a conserved manner. Olofsson at page 2577, col. 2, and FIGS. 1 and 2 therein. Although VEGF-B is secreted, it remains bound to cells and to the extracellular matrix. It is released from the cells by the addition of heparin. Id. at page 2579, col. 1. The receptors to which VEGF-B binds are unknown. Id. Olofsson suggests that VEGF-B may have one of two opposite functions, i.e., “[c]ell associated VEGF-B may act as a local growth factor and growth stimulus for endothelial cells by direct cell-cell interaction . . . Alternatively, cell association may functionally inactivate VEGF-B by making it inaccessible for endothelial cells.” Olofsson, et al., at pages 2579-2580.

VEGF-C, which is expressed most prominently in the heart, lymph nodes, placenta, ovary, small intestine and thyroid, is induced by a variety of growth factors, inflammatory cytokines and hypoxia. VEGF-C is recombinantly expressed as disclosed in Joukov et al. and has the amino acid sequence disclosed at page 291 and FIG. 3 therein. See Joukov et al., “A novel vascular endothelial growth factor, VEGF-C, is a ligand for the Flt4 (VEGFR-3) and KDR (VEGFR-2) receptor tyrosine kinases,” The EMBO Journal, 15(2):290-298 (1996); also Ferrara, et al., “The Biology of Vascular Endothelial Growth Factor,” Endocrine Reviews, 18(1):4-25 (1997) at FIG. 3. VEGF-C is the largest member of the VEGF family, having 399 amino acid residues and only 32% homology to VEGF-A. See Ferrara (1997) at page 11, col. 1. The carboxy end of VEGF-C contains 180 residues of insert (at residue positions 213-295) that are not found in the other VEGFs. See Joukov et al. (1996) at FIG. 3: or Ferrara, et al., “The Biology of Vascular Endothelial Growth Factor,” Endocrine Reviews, 18(1):4-25 (1997) at FIG. 4. In precursor form, VEGF-C has very little activity. Enholm, et al., Vascular Endothelial Growth Factor-C: A Growth Factor for Lymphanc and Blood Vascular Endothelial Cells,” Trends Cardiovasc. Med., 8(7):292-297 (1998) at page 293, col. 3. The fully processed (mature) form of VEGF-C binds to VEGFR-2 (previously known as flt-1 and KDR/Flk-1), which is a receptor expressed in blood vessels, and to VEGFR-3 (also known as Flt4), which is a receptor predominantly expressed in the lymphatic endothelium of adult tissues. See Enholm (1998) at page 293, col. 3; also Joukov et al. (1996) at page 290 (Abstract). Although VEGF-C is able to compete with VEGF-A for binding to VEGFR-2, none of the three basic amino acid residues in VEGF-A that are reported to be critical for binding to VEGFR-2 are found in VEGF-C. Enholm (1998) at page 293, col. 3 to page 294, col. 1. VEGF-C exhibits significantly different properties from VEGF-A. In particular, Enholm reports that in transgenic mice that were generated to express VEGF-C in the basal cells of the epithelia, the dermis was atrophic, its connective tissue was replaced by large lymphatic vessels, the vessels had overlapping endothelial junctions, anchoring filaments in the vessel wall and discontinuous or even absent basement membrane. Enholm (1998) at page 294, cols. 2-3. In short, Enholm reports that “the endothelial proliferation induced by VEGF-C led to hyperplasia of the superficial lymphatic network, but it did not appear to induce the sprouting of new vessels.” See Enholm (1998) at page 294, col. 3. In the avian chorioallantoic membrane (CAM) assay, a widely used angiogenesis assay, a lymphangiogenic response was observed as well as an “inconspicuous angiogenic effect at high concentrations” of VEGF-C. See Enholm (1998) at page 294, col. 3. However, the combination of VEGF-A and VEGF-C reportedly provided a synergistic effect in the induction of in vitro angiogenesis in collagen gel, wherein the effect was more prominent in cells expressing both VEGFR-2 and VEGFR-3 than in those expressing only VEGFR-2. See Enholm (1998) at page 294, col. 3, citing to Pepper, et al., “Vascular Endothelial Growth Factor (VEGF)-C synergizes with Basic Fibroblast Growth Factor and VEGF in the Induction of Angiogenesis in vitro . . . ” J. Cell Physio., (in press).

VEGF-D, which is the most recent member of the VEGF family to be discovered, is encoded by the cDNA and has the amino acid sequence shown in FIG. 2 of commonly assigned U.S. Ser. No. 09/043,476, filed Mar. 18, 1998, now pending; and corresponding WO 97/12972 which was published on Apr. 10, 1997. VEGF-D is a dimerizing protein having 354 amino acid residues. Thus, VEGF-D is substantially larger than VEGF-A (121-206 residues) and VEGF-B (167 residues), but smaller than VEGF-C (399 residues). The core of VEGF-D is highly conserved relative to the other VEGF proteins. Moreover, like the other members of the VEGF family, VEGF-D contains the 15 cysteine residues at residue positions 111, 136, 142, 145, 146, 153, 189, 191, 258, 269, 271, 273, 300, 312 and 314 that are highly conserved throughout the VEGFs, and the 8 cysteine residues that are conserved in the PDGFs. Overlapping comparisons of the amino acid sequences of the VEGFs and some of the PDGFs, showing the conserved areas, are found in Ferrara, et al., “The Biology of Vascular Endothelial Growth Factor,” Endocrine Reviews, 18(1):4-25 (1997) at FIG. 4; in WO 97/12972 and its U.S. equivalent U.S. Ser. No. 09/043,476 at FIG. 3; and WO 98/02543 at FIG. 3. Although there are conserved areas between the various species of VEGF and PDGF, there are substantial differences. VEGF-D shares 48% sequence identity with VEGF-C, its closest family member, and has similar N-terminal and C-terminal extensions, a feature which distinguishes them from other members of the VEGF family. Unlike VEGF-A, VEGF-D does not bind to VEGFR-1. Rather, like VEGF-C, VEGF-D binds to both VEGFR-2 and VEGFR-3. See Achen et al., “Vascular Endothelial Growth Factor-D (VEGF-D) is a Ligand for the Tyrosine Kinases VEGF Receptor-2 (Flk1) and VEGF Receptor-3 (Flt4),” PNAS USA 95:548-553 (1998). Thus, VEGF-D would be expected to behave similar to VEGF-C. According to Enholm, “VEGF-C and VEGF-D may regulate the lymphatic regeneration occurring in tissue and the responses of lymphatic vessels in inflammatory processes.” Enholm et al., (1998) at page 296, col. 1. Enholm further states that “[m]ajor questions about the biological roles of VEGF-C are not answered yet” such as the importance of the “potential of mature VEGF-C to induce proliferation of blood vessels via the VEGFR-2 . . . ” Id. Thus, although VEGF-C has been known since 1996, even less is known about the more recently discovered VEGF-D. Accordingly, it is an object of the present invention to discover various properties of VEGF-D and a method of their use.

### SUMMARY OF THE INVENTION

It has been discovered that Figf/VEGF-D (hereinafter “VEGF-D”) induces angiogenesis in vivo using the conventional “cornea micro-pocket assay” for angiogenesis. In addition, it has been discovered that VEGF-D induces angiogenesis in vitro in the conventional endothelial cell gel assay. Thus, in one aspect, the present invention is directed to a method for inducing angiogenesis in a tissue comprising providing a tissue in need of angiogenesis with an angiogenically effective amount of a recombinant VEGF-D or an angiogenically active fragment or mutein thereof. In an embodiment of the present invention, the VEGF-D has the amino acid sequence shown in SEQ ID NO: 1 or is an angiogenically active fragment or mutein thereof. In another embodiment, the present invention is directed to a method for inducing angiogenesis in an area in need of angiogenesis in a mammal comprising providing the area in need of angiogenesis with an angiogenically effective amount of a recombinant VEGF-D or an angiogenically active fragment or mutein thereof.

In any of the methods of the present invention, the VEGF-D is provided in solution or slow-release form. When the VEGF-D is provided in solution form, the angiogenically effective amount of said VEGF-D or said angiogenically active fragment or mutein thereof is from about 1 ng/100 ml to 32 μg/100 ml, typically from about 2 ng/100 ml to 2 μg/100 ml; more typically, from about 3 ng/100 ml to 500 ng/100 ml; most typically, from about 5 ng/100 ml to 200 ng/100 ml. When the VEGF-D is provided in slow-release form, the angiogenically effective amount of said VEGF-D or said angiogenically active fragment or mutein thereof is from about 2 ng to 2 μg; typically, from about 3 ng to 500 ng; more typically, from about 5 ng to 200 ng.

### BRIEF DESCRIPTION OF THE FIGURES

FIGS. 1A-1C show that implanted Figf/VEGF-D expressing cells induce neovascularization in rabbit corneas. FIG. 1A is a Western blot of a gel showing the relative expression of Figf/VEGF-D, a >30 kDa protein, in CHO cells by clones #65 and #79. Equal volumes of culture supernatants from the clones #65 and #79 were precipitated and analyzed on the Western blot using an anti-Figf/VEGF-D rabbit polyclonal antiserum. FIG. 1B is a plot of angiogenic score versus time (days) for CHO cells (4×10⁴) either as a mock transfectant (C) (open squares), or clone #65 (open circle) expressing low levels of Figf/VEGF-D (0.1 ng/ml protein in supernatant), or clone #79 (closed circle) expressing higher levels of Figf/VEGF-D (approximately 0.5 ng/ml protein in supernatant) that were surgically implanted into the corneas. New blood vessel growth was recorded every other day with a slit lamp stereomicroscope. Angiogenic scores were calculated on the basis of the number of vessels and their growth rate and plotted versus time (hours). Angiogenic score data are the mean values obtained from the response scored in all animals in this study (n=48). FIGS. 1C(a)-1C(d) show pictures of rabbit corneas from a representative experiment. FIG. 1C(a) shows a corneal implant of CHO mock transfectant; and FIGS. 1C(b), 1C(c) and 1C(d) show clone #79 promotes and sustains vascular growth over time at days 6, 9 and 14, respectively. Corneas were photographed with stereomicroscope. Magnification was at 18×.

FIGS. 2A-2D show that Figf/VEGF-D sustains dose-dependent angiogenesis in vivo. FIG. 2A is a Western blot showing that the supernatant of S. cerevisiae yeast strains expressing Figf/VEGF-D and Figf/VEGF-D N160P mutant (“mut N160P”) as indicated. FIG. 2B is a plot of angiogenic score versus time (days) shows the angiogenic activity of the following four concentrations of Figf/VEGF-D that were tested as slow release preparations in the rabbit cornea assay: 100 ng (open circles); 200 ng (closed circles); 300 ng (triangles); and 400 ng (diamonds). FIG. 2C is a plot of angiogenic score versus time (days) for 200 ng (diamond) and 400 ng (circle)/pellet of Figf/VEGF-D N160P. FIG. 2D is a plot of angiogenic score versus time (days) for 200 ng/pellet of VEGF-A₁₂₁ (triangle) and VEGF-A₁₆₅ (diamond) as a comparison. Angiogenic scores are calculated as consistently described in FIG. 1 and elsewhere herein. Each experiment was repeated at least four times.

FIGS. 3A-3F are photographs of HUVEC cultured in three-dimensional matrigel in low serum conditions, showing that Figf/VEGF-D induced endothelial cell morphological changes in vitro. FIG. 3A shows the control culture. FIG. 3B shows a culture to which 20 ng/ml of VEGF-A was added. FIGS. 3C, 3D, 3E and 3F show cultures to which were added 5 ng/ml, 10 ng/ml, 50 ng/ml and 100 ng/ml, respectively of Figf/VEGF-D. Photographs were taken twenty-four hours after VEGF-A or Figf/VEGF-D treatment.

FIGS. 4A-D show that Figf/VEGF-D induced tyrosine phosphorylation of VEGFR-2 and VEGFR-3 receptors. HUVEC and KS IMM cells were incubated with Figf/VEGF-D. After stimulation receptors were immunoprecipitated with anti-receptor antibody and analyzed by Western blotting with an anti-phosphotyrosine monoclonal antibody. FIGS. 4A and 4B show the Western blots of the phosphorylation of VEGFR-2 and VEGFR-3, respectively in HUVEC. FIGS. 4C and 4D phosphorylation of VEGFR-2 and VEGFR-3, respectively in KS IMM. A positive control (+) and Figf/VEGF-D stimulation (D) is indicated. Arrows denote the position of the phosphorylated 210 kDa VEGFR-2 protein and the positions of the phosphorylated, proteolytically processed 125 kDa and unprocessed 195 kDa forms of VEGFR-3.

FIGS. 5A-5D are bar graphs show that Figf/VEGF-D induced cell proliferation and chemotactic activity. FIGS. 5A and 5B show that Figf/VEGF-D caused proliferation of HUVEC and KS IMM cells, respectively in a concentration dependent manner up to about 50 ng/ml of Figf/VEGF-D. Experiments were performed in medium containing 1% FCS. After seventy-two hours, cells were enumerated using Coulter counter and values represent the mean (±SEM) of triplicate samples. FIGS. 5C and 5D show the effect of Figf/VEGF-D concentration (0 ng/ml to 300 ng/ml) on the migration of HUVEC and KS IMM cells, respectively. Cells were seeded in the upper wells of a 48-well microchemotaxis Boyden chamber and incubated for seven hours at 37° C. in medium containing 1% FCS. The lower wells contained the indicated concentrations of Figf/VEGF-D. Cells migrating through a polycarbonate membrane with a pore size of 5 μm were quantified by staining the cells with Giemsa solution and counting was performed on a light microscope of five high-power fields (100×). The results are expressed as the mean ±1 SD of three independent experiments performed in triplicate.

### DETAILED DESCRIPTION OF THE INVENTION

The present invention is directed to a method a method for inducing angiogenesis in a tissue comprising providing a tissue in need of angiogenesis with an angiogenically effective amount of a recombinant VEGF-D or an angiogenically active fragment or mutein thereof. In another embodiment, the present invention is directed to a method for inducing angiogenesis in an area in need of angiogenesis in a mammal comprising providing the area in need of angiogenesis with an angiogenically effective amount of a recombinant VEGF-D or an angiogenically active fragment or mutein thereof.

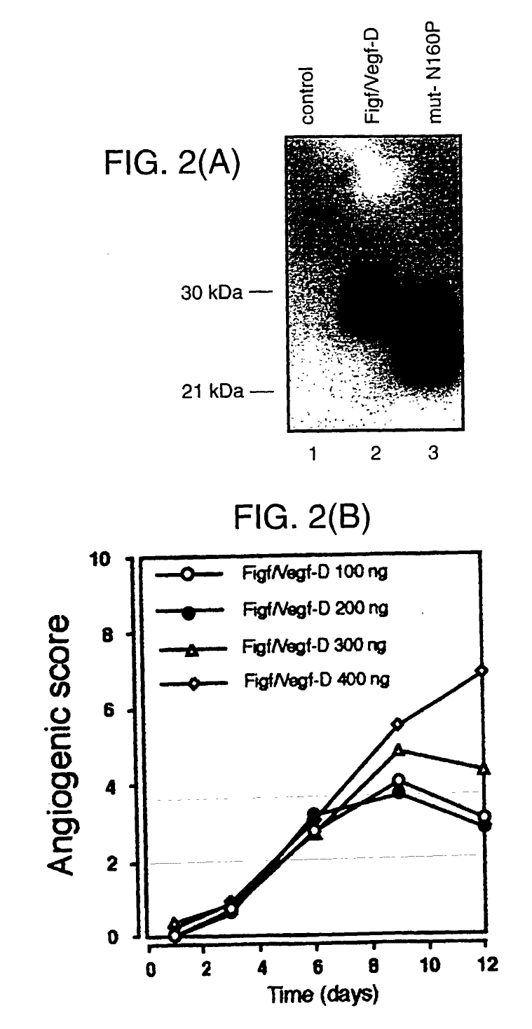

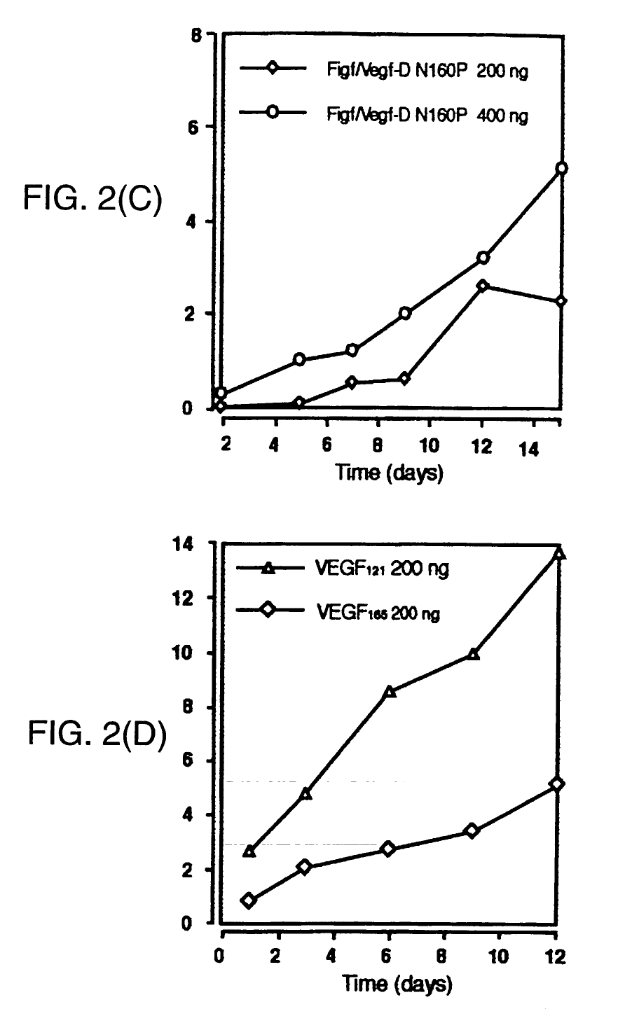

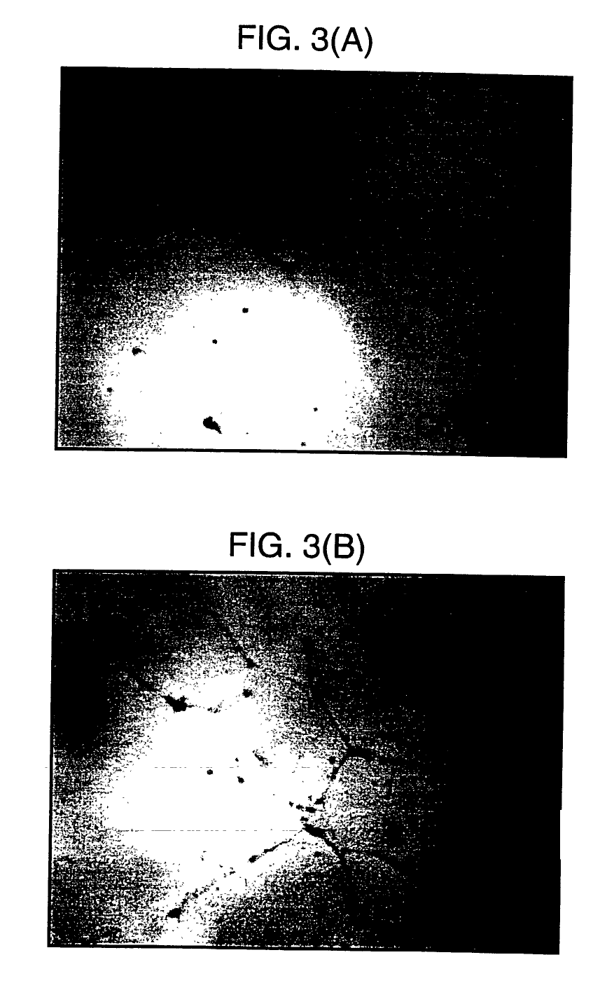

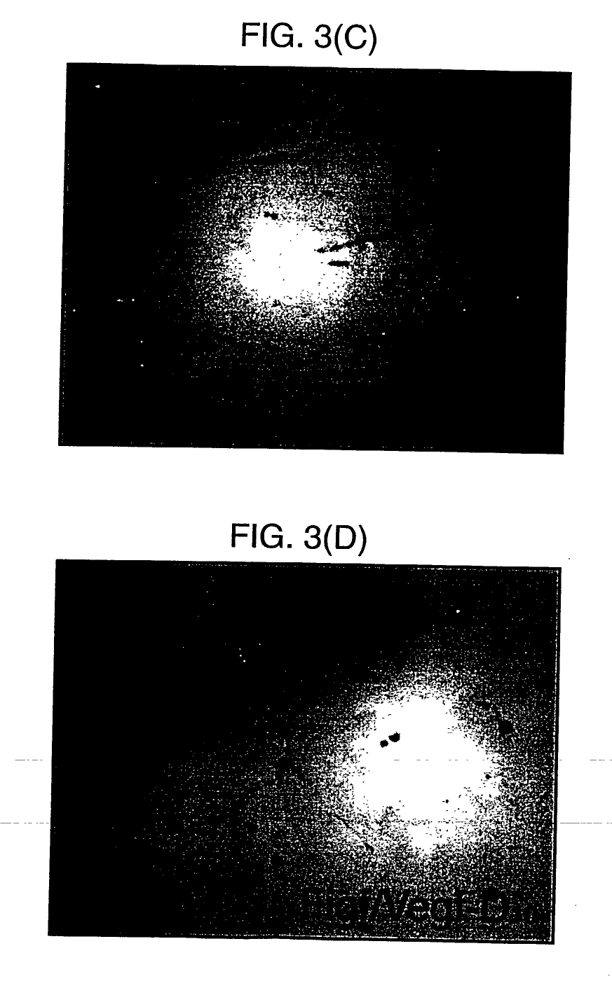

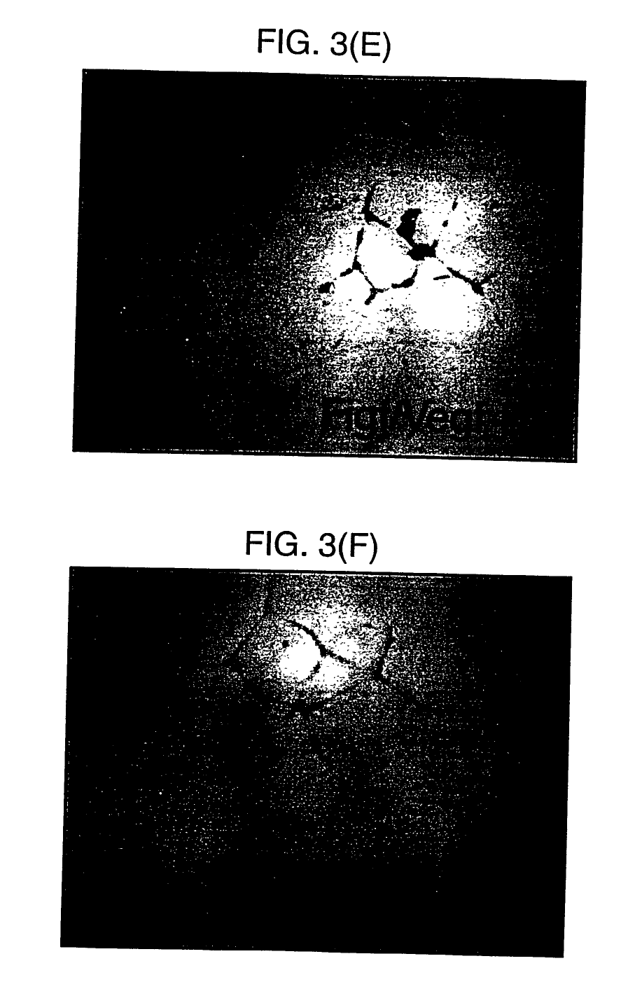

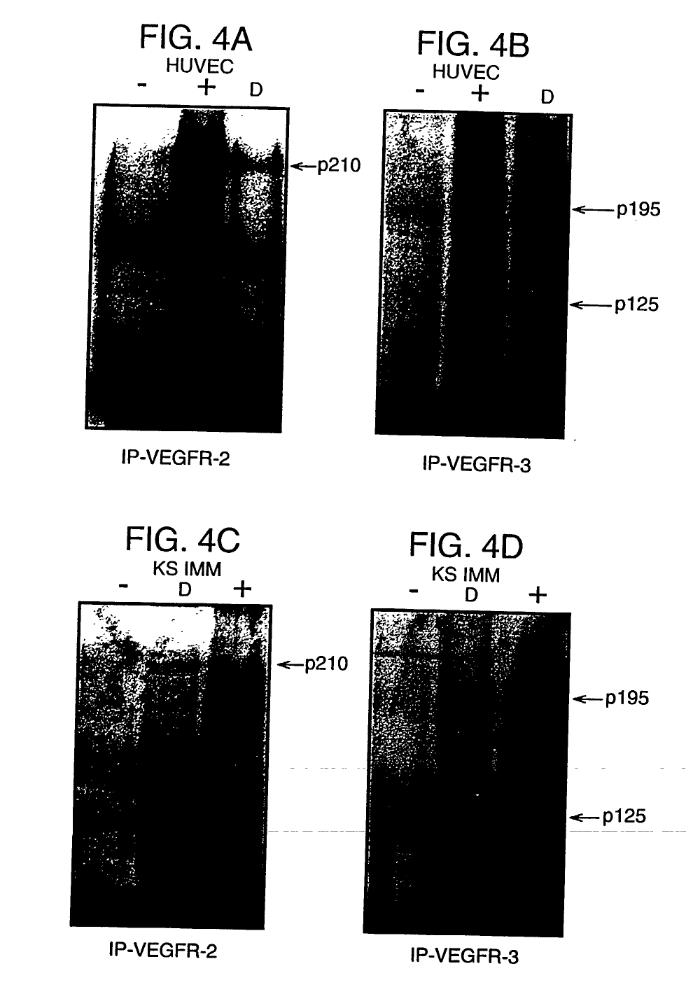

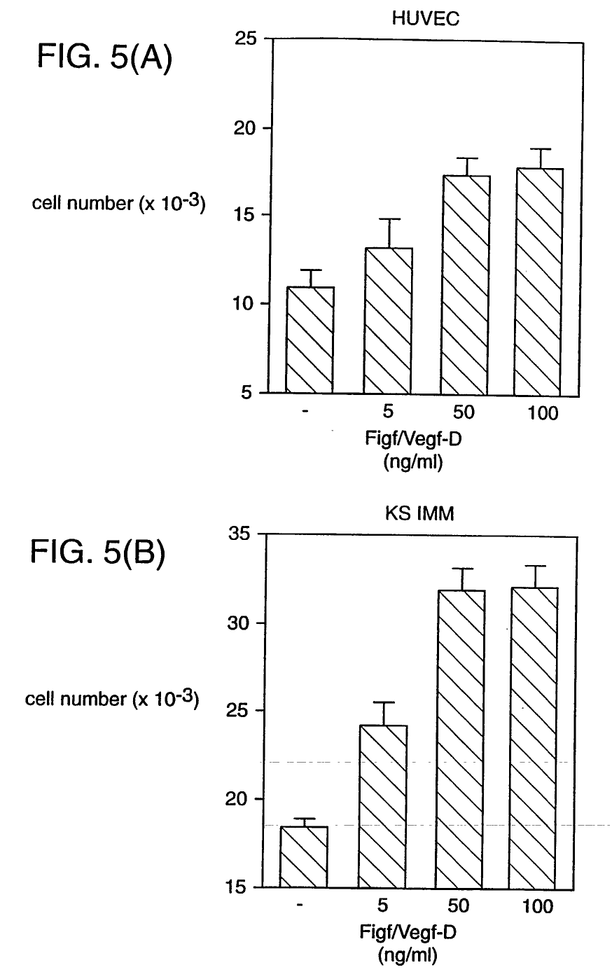

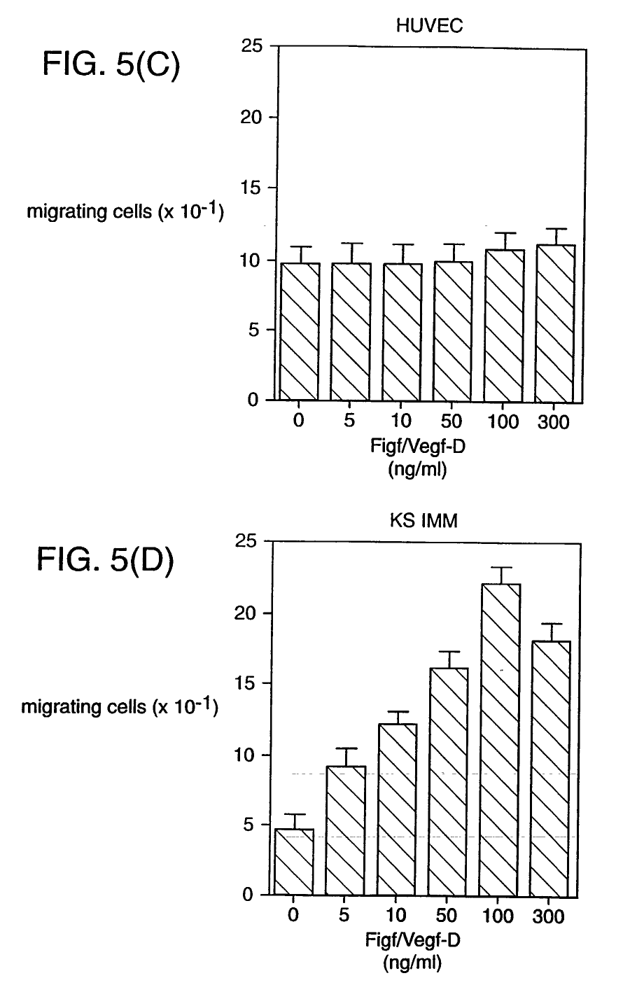

In the method of the present invention, a suitable recombinant VEGF-D is the 354 residue human mature VEGF-D having the amino acid sequence of SEQ ID NO: 1. See commonly assigned U.S. Ser. No. 09/043,476, filed Mar. 18, 1998 at FIG. 2. Another suitable VEGF-D for use in the method of the present invention was isolated from human lung and has 354 residues as disclosed in SEQ ID NO: 5 of WO 98/07832. The 354 residue VEGF-D of WO 98/07832 differs form the 354 residue VEGF-D of U.S. Ser. No. 09/043,476 and SEQ ID NO: 1 herein at 6 residue positions, i.e., 56 (Thr→Ile), 151 (Phe→Leu), 151 (Met→Ile), 261 (Asp→His), 264 (Glu→Phe) and 297 (Glu→Leu). The amino acid sequence for another suitable mature VEGF-D, having 354 amino acid residues, is disclosed as SEQ ID NO: 2 of WO 98/24811. The above-cited references and any other references cited anywhere herein are expressly incorporated herein by reference.

In addition to using VEGF-D, the method of the present invention includes the use of an angiogenic fragment thereof. By the phrase “angiogenically active fragment” of VEGF-D is meant a protein or polypeptide fragment of an angiogenic agent that exhibits at least 80% of the angiogenic activity of the parent molecule from which it was derived. WO 98/07832 discloses a naturally occurring 325 residue fragment of VEGF-D that was isolated from human breast tissue and that lacks the first 30 residues (if you include the N-terminal Met) of the mature 354 residue VEGF-D disclosed therein. Accordingly, it is within the scope of the present invention that the term “angiogenically active fragment” also include those fragments of mature VEGF-D, such as the VEGF-D of SEQ ID NO: 1, that also lack one or more of the first thirty residues from the N-terminus. Further, when the amino acid sequence of the VEGF-D of SEQ ID NO: 1 is compared to the amino acid sequence of VEGF-A₁₆₅, the VEGF-D contains an C-terminal extension of 57 residues from residue position 287. Thus, the term “angiogenically active fragment” as used herein would encompass those VEGF-D fragments having at least residues 31-287 of mature VEGF-D, such as the VEGF-D of SEQ ID NO: 1 or the other VEGF-Ds disclosed herein.

The method of the present invention also includes the use of an angiogenically active mutein of VEGF-D. By the phrase “angiogenically active mutein,” as used herein, is meant an isolated and purified recombinant protein or polypeptide that has 65% sequence identity (homology) to any naturally occurring VEGF-D, as determined by the Smith-Waterman homology search algorithm (Meth. Mol. Biol. 70:173-187 (1997)) as implemented in MSPRCH program (Oxford Molecular) using an affine gap search with the following search parameters: gap open penalty of 12, and gap extension penalty of 1, and that retains at least 80% of the angiogenic activity of the naturally occurring angiogenic agent with which it has said at least 65% sequence identity. Preferably, the angiogenically active mutein has at least 75%, more preferably at least 85%, and most preferably, at least 90% sequence identity to the naturally occurring VEGF-D. Other well-known and routinely used homology/identity scanning algorithm programs include Pearson and Lipman, PNAS USA, 85:2444-2448 (1988); Lipman and Pearson, Science, 222:1435 (1985); Devereaux et al., Nuc. Acids Res., 12:387-395 (1984); or the BLASTP, BLASTN or BLASTX algorithms of Altschul, et al., Mol. Biol., 215:403-410 (1990). Computerized programs using these algorithms are also available and include, but are not limited to: GAP, BESTFIT, BLAST, FASTA and TFASTA, which are commercially available from the Genetics Computing Group (GCG) package, Version 8, Madison Wis., USA; and CLUSTAL in the PC/Gene program by Intellegenetics, Mountain View Calif. Preferably, the percentage of sequence identity is determined by using the default parameters determined by the program.

The phrase “sequence identity,” as used herein, is intended to refer to the percentage of the same amino acids that are found similarly positioned within the mutein sequence when a specified, contiguous segment of the amino acid sequence of the mutein is aligned and compared to the amino acid sequence of the naturally occurring angiogenic agent.

When considering the percentage of amino acid sequence identity in the mutein, some amino acid residue positions may differ from the reference protein as a result of conservative amino acid substitutions, which do not affect the properties of the protein or protein function. In these instances, the percentage of sequence identity may be adjusted upwards to account for the similarity in conservatively substituted amino acids. Such adjustments are well-known in the art. See, e.g., Meyers and Miller, “Computer Applic. Bio. Sci., 4:11-17 (1988).

To prepare an “angiogenically active mutein” of a VEGF-D of the present invention, one uses standard techniques for site directed mutagenesis, as known in the art and/or as taught in Gilman, et al., Gene, 8:81 (1979) or Roberts, et al., Nature, 328:731 (1987). Using one of the site directed mutagenesis techniques, one or more point mutations would introduce one or more conservative amino acid substitutions or an internal deletion. Conservative amino acid substitutions are those that preserve the general charge, hydrophobicity/hydrophilicity, and/or steric bulk of the amino acid being substituted. By way of example, substitutions between the following groups are conservative: Gly/Ala, Val/Ile/Leu, Lys/Arg, Asn/Gln, Glu/Asp, Ser/Cys/Thr, and Phe/Trp/Tyr. Significant (up to 35%) variation from the sequence of the naturally occurring VEGF-D is permitted as long as the resulting protein or polypeptide retains angiogenic activity within the limits specified above.

Cysteine-depleted muteins are muteins within the scope of the present invention. These muteins are constructed using site directed mutagenesis as described above, or according to the method described in U.S. Pat. No. 4,959,314 (“the '314 patent”), entitled “Cysteine-Depleted Muteins of Biologically Active Proteins.” The '314 patent discloses how to determine biological activity and the effect of the substitution. Cysteine depletion is particularly useful in proteins having two or more cysteines that are not involved in disulfide formation.

In the method of the present invention, an angiogenically effective amount of VEGF-D or a fragment or mutein therof is administered or provided in a pharmaceutically acceptable carrier as a solution or as a slow-release formulation. By the term “pharmaceutically acceptable carrier” as used herein is meant any of the carriers or diluents that are well-known in the art for the stabilization and/or administration of a proteinaceous medicament that does not itself induce the production of antibodies harmful to the patient receiving the composition, and which may be administered without undue toxicity. The choice of the pharmaceutically acceptable carrier and its subsequent processing enables the composition of the present invention to be provided in either liquid (solution) or solid form.

When the pharmaceutical composition and/or unit dose composition are in liquid form, the pharmaceutically acceptable carrier comprises a stable carrier or diluent suitable for intravenous (“IV”) or intracoronary (“IC”) injection or infusion. Suitable carriers or diluents for injectable or infusible solutions are nontoxic to a human recipient at the dosages and concentrations employed, and include sterile water, sugar solutions, saline solutions, protein solutions or combinations thereof.

Typically, the pharmaceutically acceptable carrier includes a buffer and one or more stabilizers, reducing agents, anti-oxidants and/or anti-oxidant chelating agents. The use of buffers, stabilizers, reducing agents, anti-oxidants and chelating agents in the preparation of protein based compositions, particularly pharmaceutical compositions, is well-known in the art. See, Wang et al., “Review of Excipients and pHs for Parenteral Products Used in the United States,” J. Parent. Drug Assn., 34(6):452-462 (1980); Wang et al., “Parenteral Formulations of Proteins and Peptides: Stability and Stabilizers,” J. Parent. Sci. and Tech., 42:S4-S26 (Supplement 1988); Lachman, et al., “Antioxidants and Chelating Agents as Stabilizers in Liquid Dosage Forms-Part 1,” Drug and Cosmetic Industry, 102(1): 36-38, 40 and 146-148 (1968); Akers, M. J., “Antioxidants in Pharmaceutical Products,” J. Parent. Sci. and Tech., 36(5):222-228 (1988); and Methods in Enzymology, Vol. XXV, Colowick and Kaplan Eds., “Reduction of Disulfide Bonds in Proteins with Dithiothreitol,” by Konigsberg, pages 185-188. Suitable buffers include acetate, adipate, benzoate, citrate, lactate, maleate, phosphate, tartarate and the salts of various amino acids. See Wang (1980) at page 455. Suitable stabilizers include carbohydrates such as threlose or glycerol. Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers (1988) at pages 225 to 226. Suitable antioxidants include sodium bisulfite, sodium sulfite, sodium metabisulfite, sodium thiosulfate, sodium formaldehyde sulfoxylate, and ascorbic acid. See Akers (1988) at pages 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See e.g., Wang (1980) at pages 457-458 and 460-461, and Akers (1988) at pages 224-227. Suitable sugars include glycerol, threose, glucose, galactose and mannitol, sorbitol. A suitable protein is human serum albumin.

The VEGF-D of the present invention may also be administered in a slow release formulation. Carriers for such slow release formulations are well-known in the art. Such carriers include pharmaceutically acceptable liposomes, and porous resins or gels. An alternative slow release carrier would be a compatible cell line transformed with a vector to express the VEGF-D or angiogenically active fragment or mutein thereof.

The pharmaceutical compositions containing the VEGF-D or an angiogenically active fragment or mutein thereof are provided or administered so as to contact the cells, tissue or area of the body in need of angiogenesis. In an in vitro embodiment of the present invention, the VEGF-D or angiogenically active fragment or mutein thereof is placed in contact with the cells or tissue in culture, such as by pipetting into the culture a predetermined volume of the solution containing an effective amount of the VEGF-D or angiogenically active fragment or mutein thereof. Alternatively, the in vitro cells or tissue are contacted with a slow release formulation such as a porous resin having therein an angiogenically effective amount of VEGF-D or an angiogenically active fragment or mutein thereof.

In the in vivo embodiment of the present invention, the VEGF-D or angiogenically active fragment or mutein thereof is administered to the area of need using conventional techniques, such as injection, infusion or implantation. For example, when the area in need of angiogenesis is the myocardium, the VEGF-D or angiogenically effective fragment or mutein thereof is delivered to the myocardium of a patient in need of angiogenesis using any one of the art known techniques for myocardium drug delivery. The need of a patient for angiogenesis is evaluated by the treating physician using conventional evaluation techniques such as coronary angiography, MRI and the like. In its simplest embodiment, a needle attached to a drug delivery device, such as a syringe, is stereotactically directed from outside the body through the chest cavity and the pericardium to an area of the myocardium in need of angiogenesis for delivery of an angiogenically effective amount of VEGF-D or an angiogenically active fragment or mutein thereof. Once a dosage has been delivered, the needle is withdrawn or repositioned to one or more sites on the myocardium for delivery of one or more dosages, respectively, of an angiogenically effective amount of VEGF-D or an angiogenically active fragment or mutein thereof.

In another embodiment of the method for inducing angiogenesis, the angiogenically effective amount of VEGF-D or an angiogenically active fragment or mutein thereof is delivered directly into the myocardium from a device having its proximal end outside the body and its distal end positioned within a coronary vein, a coronary artery or a chamber of the heart. A plurality of devices for delivering medicaments to the heart from a coronary vein, coronary artery or from a chamber of the heart are well-known in the art. Examples of such devices include cardiac catheters having a retractable needle at the distal end, which upon being positioned adjacent an area of the myocardium in need of angiogenesis, can project the needle into the myocardium for delivery of a predetermined amount of medicament. In the present method, such a device delivers an ultra-low dose of angiogenic agent of the present invention to an area of the myocardium in need of angiogenesis. After delivery of the angiogenic agent, the needle is retracted into the distal end, and the distal end of the device is repositioned adjacent a second area of the myocardium in need of angiogenesis, whereupon the needle is again projected into the myocardium and an ultra-low dose of the angiogenic agent is delivered. This procedure is then repeated as often as needed. The needle of the above-described embodiment is also replaceable by a laser, such as used in laser angioplasty, wherein the laser is used to bore a channel into the area of the myocardium in need of angiogenesis, and an orifice adjacent the laser delivers an angiogenically effective amount of VEGF-D or an angiogenically active fragment or mutein thereof directly into the channel. This latter device is described in WO 98/05307, entitled “Transmural Drug Delivery Method and Apparatus,” and in corresponding U.S. Ser. No. 08/906,991, filed Aug. 6, 1997, and assigned to LocalMed, Palo Alto Calif. Similar cardiac catheters suitable for drug delivery are commercially available from manufacturers such as ACS, Guidant, Angion, and LocalMed.

Other devices that are suitable for delivery of a medicament to the myocardium include delivery devices having a series of drug delivery pores positioned on the outer surface of the balloon portion of a conventional balloon cardiac catheter, which upon inflating the balloon, bring the drug delivery pores in direct contact with the vascular epithelium. The medicament is then delivered through the drug delivery pores under pressure which forces the medicament past the epithelium and into the underlying myocardium. Devices of this type are disclosed in U.S. Pat. No. 5,810,767, entitled “Method and Apparatus for Pressurized Intraluminal Drug Delivery” which issued on Sep. 22, 1998; and in U.S. Pat. No. 5,713,860, entitled “Intravascular Catheter with Infusion Array” which issued on Feb. 3, 1998; and in pending application WO 97/23256, entitled “Localized Intravascular Delivery of Growth Factors for Promotion of Angiogenesis” and corresponding U.S. Ser. No. 08/753,224, now pending.

The above-described cardiac catheters are utilized using standard techniques for cardiac catheter use. Typically, the treating physician inserts the distal end of the catheter into the femoral or subclavian artery of the patient in need of coronary angiogenesis, and while visualizing the catheter, guides the distal end into a coronary artery, vein or chamber of the heart that is proximate to the area of the heart in need of angiogenesis. The distal end of the catheter is positioned adjacent an area of the myocardium in need of angiogenesis and used as described above to deliver an angiogenically effective amount of VEGF-D or an angiogenically active fragment or mutein thereof.

The above techniques for drug delivery can also be used to treat an area of the body that is in need of angiogenesis other than the heart.

In accordance with the present invention, an angiogenically effective amount of VEGF-D or an angiogenically effective fragment or mutein thereof is delivered. When the VEGF-D (or fragment or mutein) is delivered as a solution, the amount of solution delivered is typically between about 0.050 ml and about 5 ml. The amount of solution delivered depends upon the tissue in need of angiogenesis and the need of the patient as assessed by the treating physician. For example, it would be reasonable to inject 5 ml of the pharmaceutical composition into a skeletal muscle in need of angiogenesis, while a lesser volume would be reasonable for injection into the myocardium. A treating physician would be familiar with the volumes of medicament that could be injected into different organs and would adjust the injection volume accordingly. Thus, in a pharmaceutical composition that is administered in solution form in accordance with the method of the present invention, the concentration of VEGF-D or the angiogenically active fragment or mutein thereof is from about 1 ng/100 ml to 32 μg/100 ml; typically, from about 2 ng/100 ml to 2 μg/100 ml; more typically, from about 3 ng/100 ml to 500 ng/100 ml; most typically, from about 5 ng/100 ml to 200 ng/100 ml.

In absolute terms, or when the pharmaceutical composition that is administered in sustained release form in accordance with the method of the present invention, the amount of VEGF-D or the angiogenically active fragment or mutein thereof that is administered is from about 2 ng to about 2 μg; typically, from about 3 ng to 500 ng; and more typically, from about 5 ng to 200 ng.

Although an angiogenically effective amount of the VEGF-D or angiogenically active fragment or mutein that is injected into the myocardium with each repositioning of the delivery device, the total amount of angiogenic agent that is injected with multiple dosing is typically less than 10,000 ng (i.e., less than 10 μg).

In other embodiments of the above-described method, one or more doses of the angiogenic agent are administered to the appropriate areas of myocardium for several days, over a series of alternating days, for weeks or over a series of alternating weeks.

The diseases most often associated with a need for coronary angiogenesis are coronary artery disease (CAD), i.e., a disease in which one or more coronary arteries in the patient have become partially occluded, and myocardial infarction (MI), i.e., a disease in which a coronary artery has become sufficiently occluded to cause the necrosis of the downstream myocardial tissue that relied on the artery for oxygenated blood. Thus in another aspect, the present invention is also directed to a method for treating a patient for CAD or MI, comprising administering an effective amount of VEGF-D or an angiogenically active fragment or mutein thereof.

In the examples that follow, the c-fos induced growth factor/vascular endothelial growth factor D (Figf/Vegf-D) is a secreted factor of the VEGF family which binds to the vessel and lymphatic receptors VEGFR-2 and VEGFR-3. It was found that Figf/Vegf-D is a potent angiogenic factor in rabbit cornea in vivo in a dose-dependent manner. It was also found that in vitro Figf/Vegf-D induces tyrosine phosphorylation of VEGFR-2 and VEGFR-3 receptors in primary human endothelial cells (HUVEC) and in an immortal cell line derived from Kaposi's Sarcoma lesion (KS IMM). The treatment of HUVEC with Figf/Vegf-D induces dose-dependent cell growth. Figf/VEGF-D also induces HUVEC elongation and branching to form an extensive network of capillary like cords in three-dimensional matrix. In KS IMM cells, Figf/Vegf-D treatment results in dose-dependent mitogenic and motogenic activities. Taken together with the previous observations that Figf/Vegf-D expression is under the control of the nuclear oncogene c-fos, our data uncover a link between a nuclear oncogene and angiogenesis suggesting that Figf/Vegf-d may play a critical role in tumor cell growth and invasion.

### EXAMPLE 1

### Expression of Figf/Vegf-D

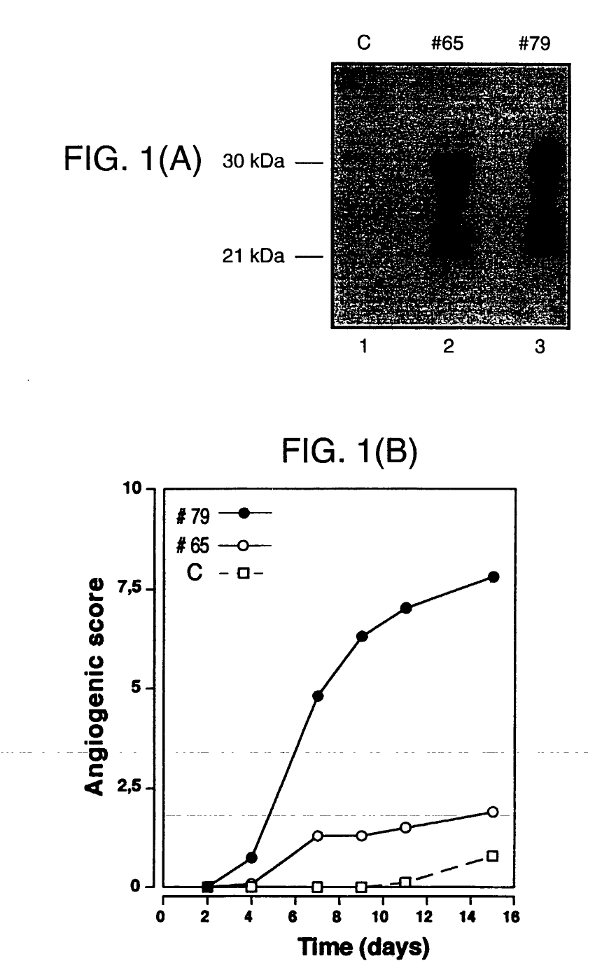

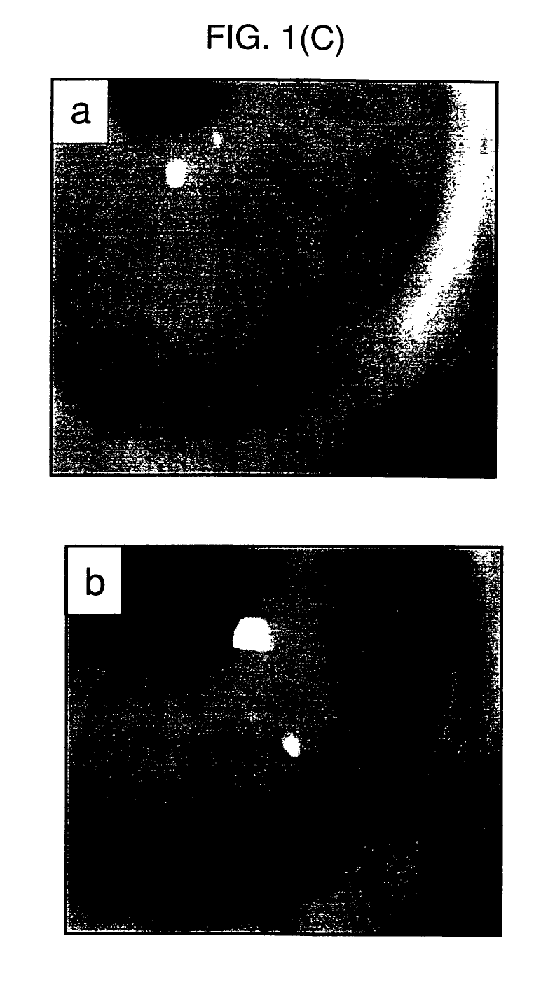

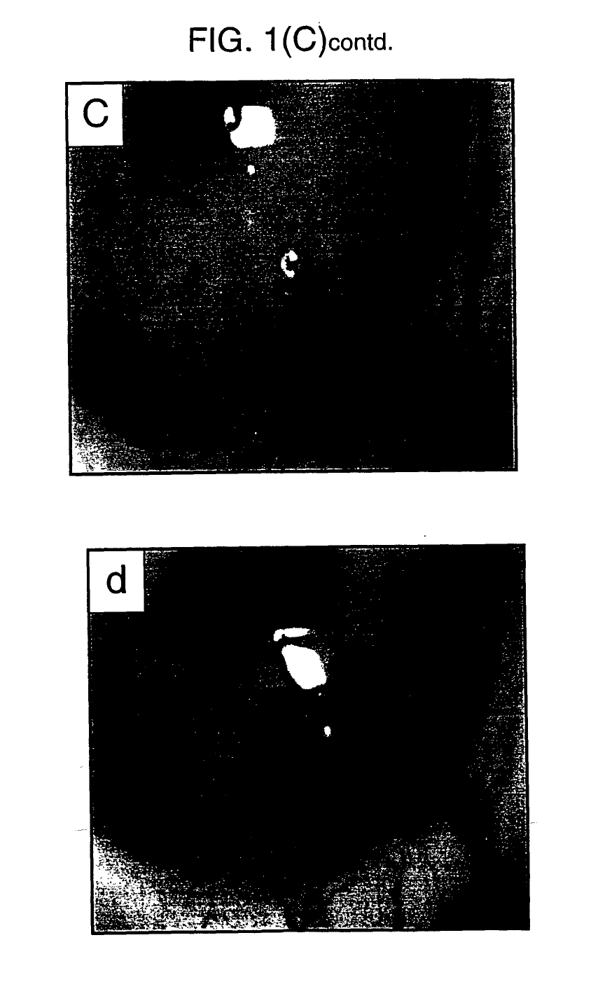

To express mature FigfVegf-D in Chinese hamster ovary (CHO) cells, the Figf/Vegf-D cDNA with a segment coding for the FLAT octapeptide (IBI/Kodak) at the C-terminus was amplified by PCR and inserted into the mammalian expression vector pcDNA3 (Invitrogen) under the control of the CMV promoter (construct LM357). CHO cells were transfected with LM357 by using calcium phosphate precipitation. Stable clones were selected in DMEM containing 10% fetal calf serum (FCS) and 800 μg/ml G418. To assay the presence of Figf/Vegf-D in CHO supernatants, isolated clones were grown in DMEM containing 2% FCS and 800 μg/ml G418 and analyzed by ELISA using anti-Figf/Vegf-D rabbit polyclonal antiserum (15). Supernatant from positive clones were precipitated with deoxycholatic acid and analyzed by Western blot. Different CHO clones expressed different Figf/Vegf-D levels. Specifically, clone 65 expressed less than 0.1 ng/ml of Figf/Vegf-D in the cell supernatant in vitro while clone 79 expressed approximately 0.5 ng/ml Figf/VEGF-D in the same conditions. To assess in vivo the angiogenic activity of increasing concentrations of the recombinant protein administered to a vascular tissue, clones 65 and 79, which express different levels of Figf/Vegf-D in vitro were selected for implantation into rabbit corneas (FIG. 1A) in the corneal micro-pocket assay of Example 2 herein.

To obtain larger amounts of purified recombinant Figf/Vegf-D, the Figf/Vegf-D was also expressed in yeast (S. cerevisiae). To obtain a secreted Figf/Vegf-D form in the yeast supernatants, the cDNA coding for the mature factor plus a segment coding for six histidine residues at the N-terminus was cloned in a yeast vector containing a secretion signal. This recombinant protein expressed in yeast was secreted into the culture medium. By contrast with the other members of the VEGF family, VEGF-C and Figf/Vegf-D contain two putative glycosylation sites in the mature protein. Secreted Figf/Vegf-D is glycosylated at asparagine 160 residue in both mammalian and in yeast cells (data not shown). To test the activity of both the glycosylated and unglycosylated forms, we also generated a Figf/Vegf-D mutant in which the glycosylation site was mutated by the introduction of a proline residue at position 160 which is present in all other known VEGF family members. Consistent with N-linked glycosylation, the wild type protein shows molecular weight increase of about 2 kDa with respect to the mutant Figf/Vegf-D N160P (FIG. 2A) and it is sensitive to endoH glycosidase (not shown).

To express mature Figf/Vegf-D in yeast, the cDNA with the coding of six histidine residues at N-terminus was amplified by PCR and inserted into the expression vector Yepsec1 immediately downstream from DNA sequence encoding the Kluyveromyces lactis toxin leader peptide (LM375) (30). The protein was expressed in S. cerevisiae yeast strain by adding galactose to the yeast culture medium since Yepsec1 construct contains a GAL “upstream activation sequence” (UASG) and the 5′ non-translated leader of the yeast CYCI gene, up to position −4 from the ATG translation initiation codon (30). An Figf/Vegf-D glycosylation mutant was obtained by PCR with the substitution N160P (LM376). Figf/Vegf-D and Figf/Vegf-D N160P proteins were purified from the yeast supernatant using a nickel column (HiTrap Chelating columns, Pharmacia Biotech) under native conditions.

### EXAMPLE 2

### In Vivo Angiogenic Assay

The angiogenic activity of Figf/Vegf-D was assayed in vivo using the rabbit cornea assay previously described (31). Corneal assays were performed in male New Zealand albino rabbits (Charles River, Calco, Lecco, Italy) in accordance with the guideline of the European Economic Community for animal care and welfare (EEC Law No. 86/609). Briefly, after being anaesthetized with sodium pentobarbital (30 mg/kg), a micro-pocket (1.5×3 mm) was surgically produced in the lower half of the cornea using pliable iris spatula 1.5 mm wide. The cell suspension (from 2.5−4×10⁵ cells/5 ml) or slow release pellets of Elvax-40 (Du-Pont) containing the purified growth factor were implanted into the micro-pocket. Subsequently, daily observation of the implants was made with a slit lamp stereomicroscope without anesthesia. An angiogenic response was scored positive when budding of vessels from the limbal plexus occurred after four days and capillaries progressed to reach the implanted pellet according to the scheme previously reported (32). The potency of angiogenic activity was evaluated on the basis of the number and growth rate of newly formed capillaries and an angiogenesis score was calculated as previously descried reported (32). Corneas were removed at the end of the experiment as well as at defined intervals after surgery and/or treatment and fixed in formalin for histological examination. A minimum of four independent experiments were performed for each condition.

Mature VEGF-C and Figf/VEGF-D factors have a molecular weight of about 30 kDa generated by proteolytic cleavage of both of the N and C-terminal domains during secretion (15, 23, 38). To obtain recombinant mature Figf/Vegf-D, we generated CHO clones by stable transfection of constructs containing the mouse Figf/Vegf-D cDNA truncated at the C-terminal proteolytic side (38). To assess in vivo the angiogenic activity of increasing concentrations of the recombinant protein administered to a vascular tissue, two clones (i.e., clones #65 and #79) expressing different levels of Figf/Vegf-D in vitro were selected for implantation into rabbit corneas (FIG. 1A). Both clone 65 and clone 79 induced corneal vascularization while the CHO mock transfectant clone did not show any angiogenic effect (FIG. 1B). Although a direct dose-response could not be made in this assay, the efficiency of the angiogenic response correlated with the amount of growth factor released in vitro as clone 79 secreted about five-fold more Figf/Vegf-D than clone 65 in the same conditions (FIG. 1A). Consistently, neovascular growth induced by clone 79 was more efficient and persisted in 100% of the implants while clone 65 did so in only 30% of corneas (FIG. 1B). This was also suggested by the direct correlation between neovascular growth observed and the number of cells implanted into corneal micro-pocket (data not shown). The angiogenic response obtained with clone 79 (FIGS. 1C(b) to 1C(d)) was comparable to the one elicited with cells expressing VEGF-A₁₂₁ (39) both in intensity and appearance.

Figf/Vegf-D that was purified to homogeneity was analyzed in the corneal micro-pocket assay in vivo. Similar to the results obtained with implanted CHO cells, purified Figf/Vegf-D induced a strong angiogenic response. After the implant of a single dose of Figf/Vegf-D in the slow release pellets, all Figf/Vegf-D doses of 100 ng/pellet to 400 ng/pellet induced capillary growth after just three days. However, a clear dose-response effect (as a function of increasing Figf/Vegf-D concentration) was evident at later time points (FIG. 2B). The Figf/Vegf-D N160P mutant showed less potent angiogenic activity with respect to the wild type protein (FIG. 2C) suggesting that Figf/Vegf-D glycosylation is either involved in receptor recognition or necessary for the correct protein folding. In this assay, recombinant Figf/Vegf-D showed intermediate activity when compared with VEGF-A₁₂₁ and VEGF-A₁₆₅ (FIG. 2D) when used at doses at 300 ng to 400 ng. Corneal angiogenesis induced by either Figf/Vegf-D or VEGF-A was non-inflammatory (not shown).

### EXAMPLE 3

### Cell Cultures

Human endothelial cells were isolated from umbilical cord vein by collagenase treatment as previously described (33), and used at passage 1-4. KS IMM cells were derived from a non-AIDS patient, and are immortalized without signs of senescence after more than 120 in vitro passages. This cell line shares common markers and similar biological behavior with typical KS “spindle cells” (34). Cells were grown on gelatin-coated plastic in medium 199 supplemented with 20% heat inactivated FCS, penicillin (100 U/ml), streptomycin (50 μg/ml), heparin (50 μg/ml) and bovine brain extract (100 μg/ml) (Life Technologies, Inc., Milano, Italy).

### EXAMPLE 4

### In Vitro Angiogenesis

Because Matrigel can induce spontaneously in vitro angiogenesis, we have tested more preparations and used batches devoid of this activity. 50 μl of Matrigel (Collaborative Research, Bedford, Mass. lot 901448) (35) was added per well of 96-well tissue culture plates and allowed to gel at 37° C. for ten minutes. Human umbilical cord vein endothelial cells (HUVEC) were starved for 24 hours in M199 with 1% FCS before being harvested in phosphate buffered saline (PBS)-EDTA. 10⁴ cells were gently added to each of triplicate wells and allowed to adhere to the gel coating for thirty minutes at 37° C. Then, the medium was replaced with indicated concentrations of Figf/Vegf-D. The plates were monitored after 24 hours and photographed with a Canon microscope. Each experiment was repeated at least three times with identical results.

### EXAMPLE 5

### Immunoprecipitation and Western Blotting

Subconfluent cultures were starved as above and then cells were stimulated with the indicated concentrations of Figf/Vegf-D for ten minutes at room temperature. Positive control was done incubating cells with 50 mM sodium orthovandadate, 100 mM H₂O₂ for twenty minutes at 37° C. After three washes with cold PBS containing 1 mM sodium orthovanadate, cells were lysed for twenty minutes on ice in 50 mM Tris-HCl, pH 7.4, 150 mM NaCl, 1 mM sodium orthovanadate, 1 mM phenylmethylsulfonylfluoride, 0.1 mM ZnCl₂, and 1% Triton. Lysates (1 mg of total proteins) were incubated at 4° C. for two hours with 100 μl of a 50% solution of protein A-SEPHAROSE (Amersham-Pharmacia Biotech, Rainham, Essex, UK) in 50 mM Tris-HCl, pH 7.4, 150 mM NaCl, and anti-VEGFR-2 or anti-VEGFR-3 antibody (C-1158, Santa Cruz Biotechnology, Santa Cruz, Calif.). Immunoprecipitates were washed four times with lysis buffer, and analyzed by 8% SDS-PAGE. Proteins were transferred onto a nylon membrane (PVDF, Millipore Corp., Bedford, Mass.) and analyzed by immunoblotting with anti-phosphotyrosine monoclonal antibody (Upstate Biotechnology, Inc., Lake Placid, N.Y.). Staining was performed by a chemiluminescence assay (ECL Amersham-Pharmacia Biotech Ranham, Essex, UK).

### EXAMPLE 6

### Cell Growth Assay

2.5×10³ human endothelial cells of KS IMM cells were plated in 96-well plates (Costar, Cambridge, Mass.) coated with gelatin (Difco Laboratories, Detroit, Mich.; 0.05%, for one hour at 220° C.) in M199 medium containing 20% FCS (Irvine, Santa Ana, Calif.). After twenty-four hours, the medium was removed and replaced with M199 containing 1% FCS with or without Figf/VEGF-D. Fresh factor (Figf/Vegf-D) was added every two days. Endothelial cell numbers were estimated after staining with crystal violet by colorimetric assay described by Keung et al. (36).

### EXAMPLE 7

### Chemotaxis Assay

Chemotaxis assays on human endothelial cells and on KS IMM were performed as previously described (33, 37) with the Boyden chamber technique using a 48-well microchemotaxis chamber. Polyvinylpyrrolidone-free polycarbonate filters (Nucleopore, Corning Costar Corp., Cambridge, Mass.) with a pore size of 5 μm were coated with 1% gelatin for ten minutes at room temperature and equilibrated in M199 supplemented with 1% FCS. Indicated concentrations of purified Figf/Vegf-D were placed in the lower compartment of a Boyden chamber. Subconfluent cultures were starved as above, harvested in PBS pH 7.4 with 10 mM EDTA, washed once in PBS and resuspended in M199 containing 1% FCS, at a final concentration of 2.5×10⁶ cells/ml. After placing the filter between lower and upper chambers, 50 μl of the cell suspension was seeded in the upper compartment. Cells were allowed to migrate for seven hours at 37° C. in a humidified atmosphere with 5% CO₂. The filter was then removed, and cells on the upper side were scraped with a rubber policeman. Migrated cells were fixed in methanol, stained with Giemsa solution (Diff-Quick, Baxter Diagnostics, Rome, Italy) and counted from five random high-power field (magnitude 100×) in each well. Each experimental point was studied in triplicate.

### References

1. Ferrara, N. & Davis-Smyth, T. (1997) Endocr. Rev. 18, 4-25.

2. Nicosia, R. F. (1998) Am. J. Pathol. 153, 11-16.

3. Bussolino, F., Mantovani, A. & Persico, G. (1997) Trends Biochem. Sci. 22, 251-256.

4. Risau, W. (1997) Nature 386, 671-674.

5. Folkman, H. (1997) EXS 79, 1-8.

6. Carmeliet, P. Ferreira, V., Breier, G., Polllefeyt, S., Kiechens, L., Gertsensein, M., Faihrig, M., Vandenboech, A., Harpal, H., Ebherardt, C., Declercq, C., Pawling, J., Moons, L., Collen, D., Risau, W. & Nagy, A. (1996) Nature 380, 435-439.

7. Ferrara, N., Carver-More, K., Chen, H., Dowd, M., Lu., O'Shea, K. S. Powell-Braxton, L., Hillian, K. J. & Moore, M. W. (1996) Nature 380, 439-442.

8. Betsholtz, C., Johnsson, A., Heldin, C.-H., Westermark, B., Lind, P., Uredea, M. S., Eddy, R., Shows, T. B., Philphott, K., Mellor, A. L., Knott, T. J. & Scott, J. (1986) Nature 320, 695-699.

9. Keck, P. J., Hauser, S. D., Krivi, G., Sanzo, K., Warren, T., Feder, J. & Connolly, D. T. (1989) Science 146, 1309-1312.

10. Leung, D. W., Cachianes, G., Kuang, W., Goeddel, D. V. & Ferrara, N. (1989) Science 246, 1306-1309.

11. Maglione, D., Guerriero, V., Viglietto, G., Delli-Bovi, P. & Persico, M. G. (1991) Proc. Natl. Acad. Sci. USA 88, 9267-9271.

12. Olofsson, B., Pajusola, K., Kaipanen, A., von Euler, G., Joukov, V., Saksela, O., Orpana, A. Petterson, R. F. Alitalo, K. & Erikson, U. (1996) Proc. Natl. Acad. Sci. USA 93, 2576-2581.

13. Grimmond, S., Lagerkrantz, J., Drinkwater, C., Sillins, G., Towson, S., Pollock, P., Gotley, D., Carson, E. Rakar, S., Nordenskjold, M., Ward, L., Hayward, N. & Weber, G. (1996) Genome Res. 6, 124-131.

14. Joukov, V., Pajusola, K., Kaipainen, A., Chilov, D., Lathinen, I., Kukk, E., Saksela, O., Kalkkinen, N. & Alitalo, K. (1996) EMBO J. 15, 290-298.

15. Orlandini, M., Marconcini, L., Ferruzzi, R. & Oliviero, S. (1996) Proc. Natl. Acad. Sci. USA 93, 11675-11680.

16. Rocchigiani, M., Lestingi, M., Luddi, A., Orlandini, M., Franco, B., Rossi, E., Ballablo, A., Zuffardi, O. & Oliviero, S. (1998) Genomics 47, 207-216.

17. Ogawa, S., Oku, A., Sawano, A., Yamaguchi, S., Yazaki, Y. & Shibuya, M. (1998) J. Biol Chem 273, 31273-31282.

18. Meyer, M., Clauss, M., Lepple-Wienhues, A., Waltenberger, J., Augustin, H. G., Ziche, M., Lanz, C., Bettner, M., Rziha, H. J. & Dehio, C. (1999) EMBO J. 18, 363-374.

19. Maglione, D., Guerriero, V., Viglietto, G., Ferraro, M. G., Aprelikova, O., Alitalo, K., Del Vecchio, S., Lei, K.-J., Chou, J. Y. & Persico, G. G. (1993) Oncogene 8, 925-931.

20. Cao, Y., Chen, H., Zhou, L., Chiang, M. K., Anand-Apte, B., Weatherbee, J. A., Wang, Y., Fang, F., Flanagan, J. G. & Tsang, M. L. (1996) J. Biol. Chem. 271, 3154-3162.

21. Cao, Y., Linen, P., Farnebo, J., Cao, R., Eriksson, A., Kumar, V., Qi, J.-H., Claesson-Welsh, L. & Alitalo, K. (1998) Proc. Natl. Acad. Sci. USA 95, 14389-14394.

22. Jeltsch, M., Kaipainen, A., Joukov, V., Meng, X., Lakso, M., Rauvala, H., Swartz, M., Fukumura, D., Jain, R. K. & Alitalo, K. (1997) Science 276, 1423, 1425.

23. Schen, M. G., Jeltsch, M., Kukk, E., Makinen, T., Vitali, A., Wilks, A. F., Alitalo, K. & Stacker, S. A. (1998) Proc. Natl. Acad. Sci USA 95, 548-553.

24. Yamada, Y., Nezu, J., Shimane, M. & Hirata, Y. (1997) Genonics 42, 483-488.

25. Jenkins, N. A., Woollatt, E., Crawford, J., Gilbert, D. J., Baldwin, M. E., Sutherland, G. R., Copeland, N. G. & Achen, M. G. (1997) Chromosome Res. 5, 502-505.

26. Avantaggiato, V., Acampora, D., Orlandini, M., Oliviero, S. & Simeone, A. (1998) Mech. Dev. 73, 221-224.

27. Kukk, E., Lymboussaki, A., Taira, S., Kaipainen, A., Jeltsch, M., Joukov, V. & Alitalo, K. (1996) Development 122, 3829-3837.

28. Enholm, B., Paavonen, K., Ristimaki, A., Kumar, V., Gunji, Y., Klefstrom, J., Kivinen, L., Laiho, M., Olofsson, B., Jukov, V., Ericsson, U. & Alitalo, K. (1997) Oncogene 14, 2475-2483.

29. Ristimaki, A., Narko, K., Enholm, B., Joukov, V. & Alitalo, K. (1998) J. Biol. Chem. 273, 8413-8418.

30. Baldari, C., Murray, J. A., Ghiara, P., Cesareni, G. & C L, G. (1987) EMBO J. 6, 229-234.

31. Ziche, M., Alessandri, G. & Gullino, P. M. (1989) Lab. Invest. 61, 629-634.

32. Ziche, M., Morbidelli, L., Choudhuri, R., Zhang, H. T., Donnini, S., Granger, H. J. & Bicknell, R. (1997) J. Clin. Invest. 99, 2625-2634.

33. Bussolino, F., Wang, J. M., Defilippi, P., Turrini, F., Sanavio, F., Edgell, C. J. S., Aglietta, M., Arese, P. & Mantovani, A. (1989) Nature 337, 471-473.

34. Albini, A., Paglieri, I., Orengo, G., Carlone, S., Aluigi, M. G., DeMarchi, R., Matteucci, C., Mantovani, A., Carozzi, F., Donini, S. & Benelli, R. (1997) AIDS 11, 713-721.

35. Kleinman, H. K., McGarvery, M. L., Hassel, J. R., Star, V. L., Cannon, F. B., Laurie, G. W. & Martin, G. R. (1986) Biochemistry 25, 312-318.

36. Keung, K., Silfer, E. & Eppenberg, V. (1989) Anal. Biochem. 182, 16-19.

37. Dejana, E., Languino, L. R., Polentarutti, N., Balconi, C., Ryckewaert, J. J., Larrieu, M. J., Mantovani, A. & Marguerie, G. (1985) J. Clin. Invest. 75, 11-18.

38. Joukov, V., Sorsa, T., Kumar, V., Jeltisch, M., Claesson-Welsh, L., Cao, Y., Saksela, O., Kalkkinnen, N. & Alitalo, K. (1997) EMBO J. 16, 3898-3911.

39. Morbidelli, L., Birkenhaeger, R., Roecki, W., Granger, H. J., Kaerst, U., Welch, H. & Ziche, M. (1997) Angiogenesis 1, 117-130.

40. Montesano, R., Orci, L. & Vassalli, P. (1983) J. Cell. Biol. 97, 1648-1652.

41. Grant, D. S., Gashiro, K., Segui-Real, B., Yamada, Y., Martin, G. R. & Kleinman, H. K. (1989) Cell 58, 933-943.

42. Basilico, C. & Moscatelli, D. (1992) Adv. Cancer Res. 59, 115-165.

43. Ziche, M., Maglione, D., Ribatti, D., Mobidelli, L., Lago, C. T., Battisti, M., Paoletti, I., Barra, A., Tucci, M., Parise, G., Vincenti, V., Granger, H. J., Viglietto, G. & Persico, M. G. (1997) Lab. Invest. 76, 517-531.

44. Szebenyi, G. & Fallon, J. F. (1999) Int. Rev. Cytol. 185, 45-106.

45. Seghezzi, G., Patel, S., Ren, C. J., Gualandris, A., Pintucci, G., Robbins, E. S., Shapiro, R. L., Galloway, A. C., Rifkin, D. B. & Mignatti, P. (1998) J. Cell Biol. 141, 1659-1673.

46. Mima, T., Ueno, H., Fischman, D. A., Williams, L. T. & Mikawa, T. (1995) Proc. Natl. Acad. Sci. USA 92, 467-471.

47. Peters, K., Werner, S., Liao, X., Wert, S., Whitsett, J. & Williams, L. (1994) EMBO J. 13, 3296-3301.

48. Soker, S., Takashima, S., Miao, H. Q., Neufeld, g. & Klagsbrun, M. (1982) Cell 92, 735-75.

49. Soldi, R., Mitola, S., Strasly, S., Defilippi, P., Tarone, G. & Bussolino, F. (1999) Embo J. 18, 882-892.

50. Johnson, R. S Spiegelman, B. M. & Papaioannou, V. E. (1992) Cell 71, 577-586.

51. Wang, Z.-Q., Ovitt, C., Gregoriadis, A. E., Möhle-Steinlein, U., Rüther, U. & Wagner, E. F. (1992) Nature 360, 742-745.

52. Saez, E., Rutemberg, S. E., Mueller, E., Oppenheim, H., Smoluk, J., Yuspa, S. H. & Spiegelman, B. M. (1995) Cell 82, 721-732.

What is claimed is:

1. A method for inducing angiogenesis in a tissue comprising providing a tissue in need of angiogenesis with an angiogenically effective amount of a recombinant VEGF-D or an angiogenically active fragment or mutein thereof.

2. The method of claim 1, wherein the VEGF-D has the amino acid sequence shown in SEQ ID NO: 1.

3. The method of claim 1, wherein said VEGF-D or said angiogenically active fragment or mutein thereof is provided in solution form.

4. The method of claim 3, wherein the angiogenically effective amount of said VEGF-D or said angiogenically active fragment or mutein thereof is from about 1 ng/100 ml to 32 μg/100 ml.

5. The method of claim 4, wherein the angiogenically effective amount of said VEGF-D or said angiogenically active fragment or mutein thereof is from about 2 ng/100 ml to 2 μg/100 ml.

6. The method of claim 5, wherein the angiogenically effective amount of said VEGF-D or said angiogenically active fragment or mutein thereof is from about 3 ng/100 ml to 500 ng/100 ml.

7. The method of claim 6, wherein the angiogenically effective amount of said VEGF-D or said angiogenically active fragment or mutein thereof is from about 5 ng/100 ml to 200 ng/100 ml.

8. The method of claim 1, wherein said VEGF-D or said angiogenically active fragment or mutein thereof is provided in a slow release form.

9. The method of claim 8, wherein said angiogenically effective amount of said VEGF-D or said angiogenically active fragment or mutein thereof is from about 2 ng to 2 μg.

10. The method of claim 9, wherein said angiogenically effective amount of said VEGF-D or said angiogenically active fragment or mutein thereof is from about 3 ng to 500 ng.

11. The method of claim 10, wherein said angiogenically effective amount of said VEGF-D or said angiogenically active fragment or mutein thereof is from about 5 ng to 200 ng.

12. A method for inducing angiogenesis in an area in need of angiogenesis in a mammal comprising providing the area in need of angiogenesis with an angiogenically effective amount of a recombinant VEGF-D or an angiogenically active fragment or mutein thereof.

13. The method of claim 12, wherein the VEGF-D has the amino acid sequence shown in SEQ ID NO: 1.

14. The method of claim 12, wherein said VEGF-D or said angiogenically active fragment or mutein thereof is provided in solution form.

15. The method of claim 12, wherein the angiogenically effective amount of said VEGF-D or said angiogenically active fragment or mutein thereof is from about 1 ng/100 ml to 32 μg/100 ml.

16. The method of claim 15, wherein the angiogenically effective amount of said VEGF-D or said angiogenically active fragment or mutein thereof is from about 2 ng/100 ml to 2 μg/100 ml.

17. The method of claim 16, wherein the angiogenically effective amount of said VEGF-D or said angiogenically active fragment or mutein thereof is from about 3 ng/100 ml to 500 ng/100 ml.

18. The method of claim 17, wherein the angiogenically effective amount of said VEGF-D or said angiogenically active fragment or mutein thereof is from about 5 ng/100 ml to 200 ng/100 ml.

19. The method of claim 18, wherein said VEGF-D or said angiogenically active fragment or mutein thereof is provided in a slow release form.

20. The method of claim 19, wherein said angiogenically effective amount of said VEGF-D or said angiogenically active fragment or mutein thereof is from about 2 ng to 2 μg.

21. The method of claim 20, wherein said angiogenically effective amount of said VEGF-D or said angiogenically active fragment or mutein thereof is from about 3 ng to 500 ng.

22. The method of claim 21, wherein said angiogenically effective amount of said VEGF-D or said angiogenically active fragment or mutein thereof is from about 5 ng to 200 ng.
